(19) United States
(12) Patent Application Publication
(10) Pub. No.: US 2024/0132694 A1
(43) Pub. Date: Apr. 25, 2024

(54) POLYMERISATION

(71) Applicant: Poseidon Plastics Limited, Redcar (GB)

(72) Inventors: Martin Atkins, Redcar (GB); Nicholas Curry, Redcar (GB); Steve Evans, Redcar (GB)

(21) Appl. No.: 18/277,022

(22) PCT Filed: Feb. 14, 2022
(86) PCT No.: PCT/EP22/53544

§ 371 Date: Aug. 11, 2023

(30) Foreign Application Priority Data
Feb. 12, 2021 (GB) 2102039.1

(51) Int. Cl. C08J 11/24; C08G 63/183; C08G 63/80; C08G 63/86

(52) U.S. Cl. CPC C08J 11/24; C08G 63/183; C08G 63/80; C08G 63/866; C08J 2367/02

(57) ABSTRACT

The present invention relates to a method for preparing polymers, in particular to a method for preparing a polymer comprising constitutional units derived from bis(2-hydroxyethyl) terephthalate (BHET). The BHET used in the method is a high quality recycled BHET product, which allows plastic preparation methods in which the BHET is used to be simplified.

### FIELD OF THE INVENTION

The present invention relates to a method for preparing polymers, in particular to a method for preparing a polymer comprising constitutional units derived from bis(2-hydroxyethyl) terephthalate (BHET). The BHET used in the method is a high quality recycled BHET product, which allows plastic preparation methods in which the BHET is used to be simplified.

### BACKGROUND TO THE INVENTION

PET is a thermoplastic polymer that is used in a wide range of materials due to its properties of, among others, strength, mouldability and moisture impermeability. Common uses of PET include in packaging (e.g. in drinks bottles and food containers), in fibres (e.g. in clothing and carpets) and in thin films.

Virgin PET may be readily prepared using ethylene glycol and a terephthalate-containing monomer. Nevertheless, since its raw materials are obtained from non-renewable sources such as crude oil, there is an increasing awareness of the need to recycling PET.

When PET waste is made up of just a single type of PET, such as clear plastic water bottles, recycling may be as simple as melting and remoulding flakes of the waste material. It is, however, usual for waste to comprise a variety of different PET materials, such as a range of different coloured bottles which, if melted and remoulded, would give a product with a low visual grade. Such materials may be suitable for use in carpet fibres, but they are generally not suitable for use in packaging such as in clear water bottles.

Accordingly, there is a need for methods for recycling waste PET into a product which can be used in applications which require a high visual grade.

More sophisticated methods for recycling PET involve depolymerising the waste material to obtain, usually after a number of purification and separation steps, viable raw materials for use in the preparation of a polymer.

For instance, PET may be depolymerised using a glycolysis agent such as ethylene glycol to form BHET monomers. However, conventional methods for depolymerising PET tend to produce BHET monomers at a yield of less than 80%, with significant amounts of oligomers of BHET, in particular dimers and trimers, produced from the remainder of the PET.

Since the presence of dimers and trimers reduces the quality of a polymer that is prepared from the BHET raw material, it is conventional to purify a depolymerisation mixture in order to remove these components. Further purification is particularly important where high quality recycled PET is required, for instance recycled PET that is suitable for use in transparent and colour-free bottles.

Colour spaces are often used to denote the grade of a polymer, with the b[h] value—a measure of blue (negative values) to yellow (positive values) tone—taken as a key indicator of quality. Poor quality recycled PET typically exhibits an unwanted yellow hue.

There are a number of drawbacks associated with processes in which a depolymerisation mixture is produced which contains significant quantities of dimer and trimer. One of the most significant is that considerable amounts of the PET raw material are lost from the recycling process when it is removed in the form of dimers and trimers. Unless the dimers and trimers are recycled for further depolymerisation, which in itself requires time and energy, the efficiency of typical PET recycling processes is therefore quite low.

Other impurities are also found in the BHET that is produced using traditional PET recycling methods. One of these is isophthalic acid (IPA). IPA is often used in the preparation of PET to disrupt the crystallinity of the polymer. This enhances the mouldability of the polymer as compared to a PET homopolymer. The amount of IPA that is added will depend on the end use of the PET. For instance, in carbonated drinks bottles, IPA is typically added to the monomer mixture in an amount of from 1 to 3% by weight. In PET films, IPA is typically added to the monomer mixture in an amount of up to 20% by weight.

Recycled PET materials typically have IPA entrained therein. For instance, in the mechanical recycling of PET, all of the IPA resides in the remelted PET product, known as mechanical rPET. Due to the structural similarities between IPA and BHET, depolymerisation PET recycling methods typically produce a BHET product which also has IPA entrained therein. The amount of IPA in recycled BHET will vary depending on composition the waste PET that feeds the recycling process.

Before and/or during polymerisation of BHET obtained from depolymerising PET, the amount of IPA must be therefore be measured. If the level of IPA in the recycled BHET is above that required in the eventual PET product, the recycled BHET must either by purified further to remove IPA or blended with virgin PET to form a blend with a lower IPA level. If, however, the level of IPA in the recycled BHET is below that required in the eventual PET product, then IPA must be added to the recycled BHET. These analysis and processing steps require time and energy, further reducing the efficiency of both methods for producing recycled BHET and methods for producing polymers therefrom.

Accordingly, there is a need for improved methods for the depolymerisation recycling of waste PET. In particular, there is a need for methods for the depolymerisation recycling of waste PET which provide products suitable for use in simplified polymerisation processes.

### SUMMARY OF THE INVENTION

It has surprisingly been found that, by using a series of depolymerisation reactors, a depolymerised mixture may be obtained which contains a very high proportion of BHET monomer and relative low amounts of dimer and trimer, thereby enabling conventional purification steps in which dimers and trimers are removed to be omitted. This means that solvents that would have previously been rejected as unsuitable for further processing of the crude BHET monomer may be used.

The present inventors have found that protic solvents are highly effective for recrystallising the crude depolymerisation product. In particular, water is preferred for this use, as dimers and trimers of BHET are insoluble in water. Thus, the BHET dissolves to form an aqueous phase, while the dimers and trimers remain as solid materials which can be separated from the aqueous phase, e.g. by filtration, before recrystallisation, resulting in a high purity monomer product.

It has also surprisingly been found that a PET recycling method may be carried out which produces a BHET product which is free from PA. This enables subsequent polymerisation processes in which the recycled BHET product of the present invention is used to be simplified.

Accordingly, the present invention provides a method for preparing a polymer comprising constitutional units derived from bis(2-hydroxyethyl) terephthalate (BHET). The method comprises carrying out a polymerisation reaction using a recycled BHET product, the recycled BHET product comprising isophthalic acid (IPA) in an amount of up to 0.5% by weight.

The present invention further provides the use of a recycled BHET product in the preparation of a polymer comprising ethylene terephthalate monomer. The recycled BHET product comprises IPA in an amount of up to 0.5% by weight.

### BRIEF DESCRIPTION OF THE DRAWINGS

FIG. 1 is a graph showing the efficiency of depolymerisation reactions carried out using different series of reactors.

FIG. 2 shows photos of BHET samples which are untreated and treated with various decolourising agents, as well as pictures of PET prepared using the samples.

FIG. 3 is a diagram of an apparatus for producing a recycled BHET product from waste PET. The apparatus includes a series of three depolymerisation units (10) for depolymerising PET to form BHET; a crystallisation unit (12) for receiving the depolymerised mixture and which is suitable for crystallising a precipitate comprising BHET from the depolymerised mixture; a vessel (14) for receiving the precipitate and which is suitable for dissolving the precipitate in methanol to form a solution comprising BHET; an impurity removal unit (16) for receiving the solution comprising BHET and which removes impurities from the solution to form a purified solution; and a crystallisation unit (18) for receiving the purified solution which is suitable for crystallising a purified product comprising BHET from the purified solution.

FIG. 4 is a photo of representative waste that may be processed using the apparatus shown in FIG. 3.

FIG. 5 is a diagram of an apparatus for producing a recycled BHET product from waste PET. The apparatus includes a series of two depolymerisation units (100) for depolymerising PET to form BHET; a crystallisation unit (112) for receiving the depolymerised mixture and which is suitable for crystallising a precipitate comprising BHET from the depolymerised mixture; a vessel (114) for receiving the precipitate and which is suitable for dissolving the precipitate in water to form a solution comprising BHET; an impurity removal unit (116) for receiving the solution comprising BHET and which removes impurities from the solution to form a purified solution; and a crystallisation unit (118) for receiving the purified solution which is suitable for crystallising a purified product comprising BHET from the purified solution.

### DETAILED DESCRIPTION OF THE INVENTION

The present invention provides a method for preparing a polymer comprising constitutional units derived from bis(2-hydroxyethyl) terephthalate (BHET). A polymeric constitutional unit derived from BHET has the structure:

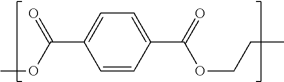

The method of the present invention comprises carrying out a polymerisation reaction using a recycled BHET product. BHET is a monomer having the following structure:

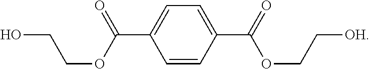 [SMILES: O=C(OCCO)c1ccc(C(=O)OCCO)cc1]

Since the BHET product used in the polymerisation reaction of the present invention is a recycled BHET product, it will typically comprise impurities which are not present in virgin BHET. One of these impurities is isophthalic acid (IPA). IPA is a monomer having the following structure:

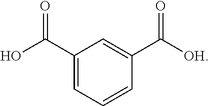 [SMILES: O=C(O)c1cccc(C(=O)O)c1]

The recycled BHET product used in the present invention advantageously comprises IPA in limited amounts, specifically in an amount of up to 0.5%. Preferably, the recycled BHET product comprises IPA in an amount of up to 0.2%, and more preferably in an amount of up to 0.1%, by weight. The amount of IPA in the recycled BHET product may be determined using standard techniques, such as nuclear magnetic resonance (NMR). NMR may be carried out using the method described below in connection with the purified BHET product.

By virtue of it being recycled, the recycled BHET product will contain trace amounts of IPA (an IPA “fingerprint”), e.g. IPA in an amount which is detectable using standard techniques, such as NMR. For instance, the recycled BHET product may contain IPA in an amount of at least 0.0001% by weight.

In preferred embodiments, the recycled BHET product is obtainable, and preferably obtained, from a PET recycling process. In some embodiments, the method of the present invention comprises providing the recycled BHET product by carrying out a PET recycling process.

The PET recycling process preferably comprises:
(a) depolymerising PET in the presence of ethylene glycol and a catalyst system in a series of depolymerisation reactors to form a depolymerised mixture comprising BHET;
(b) crystallising a precipitate comprising BHET by removing a volatiles stream comprising ethylene glycol from the depolymerised mixture using evaporation crystallisation;
(c) dissolving the precipitate in a protic solvent to form a solution comprising BHET;
(d) removing impurities from the solution to form a purified solution comprising BHET; and
(e) crystallising a purified product comprising BHET from the purified solution.

This method beneficially gives a purified BHET product with a very low IPA concentration.

The purified product comprising BHET that is produced in the PET recycling process is the recycled BHET product that is used in the polymerisation reaction of the present invention.

PET is a thermoplastic polymer having the following structure:

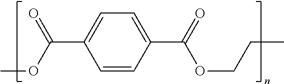

The PET that is used in the PET recycling method will typically be waste PET. The waste PET may be obtained from a wide range of sources, including packaging, bottles and textiles. Preferably the PET is obtained from waste bottles. The PET that is used in step (a) may be washed PET, i.e. PET that has been through a cleaning process. The washed PET may be PET that has been washed with water, purified by steaming, solvent cleaned and/or detergent cleaned. Preferably, the PET that is used in step (a) is PET that has been washed with water.

The PET that is used in step (a) preferably contains coloured PET. The PET may contain coloured PET in an amount of at least 5%, preferably at least 10%, and more preferably at least 25% by weight. In some embodiments, the PET may contain coloured PET in an amount of at least 50%, and more preferably at least 75% by weight. The PET may contain coloured PET in an amount of up to 100% by weight.

The PET that is used in step (a) preferably exhibits a b[h] value (i.e. a b-value on the Hunter Lab colour space) of greater than 5, for instance greater than 10, though some PET feeds may have a b[h] value of 100 or even higher. This may be measured using standard techniques, such as with a colour meter.

As the PET that is used in step (a) is typically waste PET, it will comprise constitutional units derived from IPA. The PET may comprise constitutional units derived from IPA in an amount of at least 0.5%, preferably at least 0.8%, and more preferably at least 1% by weight. The PET may comprise constitutional units derived from IPA in an amount of up to 30%, preferably up to 20%, and more preferably up to 10% by weight. Thus, the PET may comprise constitutional units derived from IPA in an amount of from 0.5 to 30%, preferably from 0.8 to 20%, and more preferably from 1 to 10% by weight. The amount of constitutional units derived from IPA in PET may be determined using standard techniques, such as nuclear magnetic resonance (NMR).

The PET is preferably used in step (a) the form of particles, such as flakes. Preferably, at least 80% by weight of the particles (i.e. d80) pass through a mesh having openings with a diameter of 20 mm, preferably 15 mm, and more preferably 12 mm. Even lower mesh sizes may also be used. Particles having these sizes are rapidly depolymerised.

Although a range of particle sizes will typically be used in step (a), larger particle sizes are preferably avoided since they may take longer to process. Accordingly, 100% by weight of the particles (d100) preferably pass through a mesh having openings with a diameter of mm, preferably 20 mm, and more preferably 12 mm. Even lower mesh sizes may also be used. Overly small particles are also preferably avoided, unless the powders are already available through waste collection and separation processes, since the energy and therefore cost required to comminute the PET to this size is unnecessary. Thus, it is preferred that a maximum of 1% by weight of the particles pass through a mesh having openings with a diameter of 0.1 mm, preferably 0.5 mm, and more preferably 1 mm.

It will be appreciated that the PET that used in step (a) may be passed to the series of reactors in a form in which it is coated with a liquid, e.g. residual water or other solvent that has been used to clean the PET. This liquid coating is not considered to form part of the PET for the purposes of the present invention.

In step (a) of the method, PET is depolymerised in a series of depolymerisation reactors to form a depolymerised mixture comprising BHET.

The PET is partially depolymerised in a first depolymerisation reactor, and further depolymerised downstream of the first reactor in the series of reactors. By using a series of reactors, it has been found that the depolymerised mixture may comprise a high proportion of BHET, and a low level dimers and trimers. Dimers and trimers have the following structure:

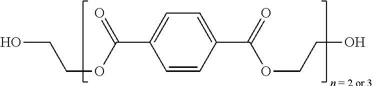

Higher oligomers will generally not be present in the depolymerised mixture. Thus, in preferred embodiments, the depolymerised mixture is substantially free from higher oligomers (i.e. where n>4).

Surprisingly, a very high quality product may be produced by depolymerising the PET in a series of just two reactors. Thus, in preferred embodiments, the PET is depolymerised in a series of two depolymerisations reactors. This gives high levels of both conversion of the PET and selectivity for BHET. In alternative embodiments, the PET is depolymerised in a series of three, or alternatively four or more, reactors.

Preferably, all of the ethylene glycol and catalyst system used in the depolymerisation process are added to the first reactor of the series. However, in some embodiments, further ethylene glycol and/or catalyst system may be added to the reaction mixture downstream of the first reactor as it is passed through the series of depolymerisation reactors.

It will be appreciated that, though ethylene glycol and/or catalyst system may be added to the reaction mixture downstream of the first reactor, no components are removed from the reaction as it passes through the series of reactors.

Each of the depolymerisation reactors used in step (a) may be operated at a temperature of at least 150° C., preferably at least 170° C., and more preferably at least 190° C. Each of the depolymerisation reactors used in step (a) may be operated at a temperature of up to 230° C., preferably up to 220° C., and more preferably up to 210° C. Thus, each of the depolymerisation reactors used in step (a) may be operated at a temperature of from 150 to 230° C., preferably from 170 to 220° C., and more preferably from 190 to 210° C. Generally, the depolymerisation reactors will be operated at the same temperature but this is not necessarily the case.

Unlike many prior art processes, the PET is preferably not used in a molten state in step (a), meaning that the reaction mixture is relatively viscous. This viscosity has typically led to relatively low levels of PET conversion. It is surprising that, by using a series of depolymerisation reactors, excellent levels of conversion can be obtained even where step (a) is carried out with PET in a solid state.

Each of the depolymerisation reactors used in step (a) may be operated at atmospheric pressure, i.e. without the application or removal of pressure. Standard atmospheric pressure is defined as 101,325 Pa. However, since atmospheric pressure varies from location to location, atmospheric pressure as used herein is considered to be approximately equal to standard atmospheric pressure, i.e. approximately 101,325 Pa.

Each of the depolymerisation reactors used in step (a) may be operated for a period of at least 20 minutes, preferably at least 45 minutes, and more preferably at least 1 hour. Each of the depolymerisation reactors used in step (a) may be operated for a period of up to 3 hours, preferably up to 2 hours, and more preferably up to 1.5 hours. Thus, each of the depolymerisation reactors used in step (a) may be operated from 20 minutes to 3 hours, preferably from 45 minutes to 2 hours, and more preferably from 1 to 1.5 hours. The depolymerisation reactors may all be operated for the same period, but this is not necessarily the case.

PET may be passed to the series of depolymerisation reactors at a flow rate of at least 1,000 kg, preferably at least 3,000 kg, and more preferably at least 5,000 kg, per hour.

PET may be passed to the series of depolymerisation reactors at a flow rate of up to 100,000 kg, preferably up to 50,000 kg, and more preferably up to 10,000 kg, per hour.

Thus, PET may be passed to the series of depolymerisation reactors at a flow rate of from 1,000 to 100,000 kg, preferably from 3,000 to 50,000 kg, and more preferably from 5,000 to 10,000 kg, per hour.

Each of the depolymerisation reactors used in step (a) is preferably operated with agitation, such as with stirring or baffles. Each reactor is preferably agitated with baffles.

Each of the depolymerisation reactors used in step (a) may comprise a grid plate or a conical base at the bottom of the reactor where solids (e.g. metals, PVC) may drop down for removal through a draw off point.

The size of the reactors used in the series of depolymerisation reactors may vary depending on how many reactors are used. Each of the reactors used in step (a) may have a size of at least 3 m³, preferably at least 8 m³, and more preferably at least 10 m³. Each of the reactors used in step (a) may have a size of up to 50 m³, preferably up to 20 m³, and more preferably up to 15 m³. Thus, each of the reactors used in step (a) may have a size of from 3 to 50 m³, preferably from 8 to 20 m³, and more preferably from 10 to 15 m³. The use of reactors on this small scale is made possible by having a series of reactors through which PET may be depolymerised with minimal residence time. Thus, industrial scale amounts of PET may be depolymerised into a high quality product using relatively small reactors.

Ethylene glycol is used in step (a) as a glycolysis agent. Ethylene glycol may be used in step (a) in amount of at least 2 times, preferably at least 3 times, and more preferably at least 3.5 times the amount of PET by weight. Ethylene glycol may be used in step (a) in amount of up to 6 times, preferably up to 5 times, and more preferably up to 4.5 times the amount of PET by weight. Thus, ethylene glycol may be used in step (a) in amount of from 2 to 6 times, preferably from 3 to 5 times, and more preferably from 3.5 to 4.5 times the amount of PET by weight.

At least 60%, preferably at least 80%, and more preferably at least 95% by weight of the ethylene glycol may be added to the first reactor. However, as mentioned above, all of the ethylene glycol is most preferably added to the first reactor. It will be appreciated that, where less than 100% of the ethylene glycol is added to the first reactor, the remainder is added to the series of depolymerisation reactors downstream of the first depolymerisation reactor.

Preferably, the ethylene glycol is heated before it is added to the series of depolymerisation reactors. Pre-heating of the ethylene glycol may be performed in a heat exchanger, for example a shell-and-tube heat exchanger which preferably uses steam as the heating medium. The ethylene glycol may be heated to a temperature of at least 150° C., preferably at least 170° C., and more preferably at least 190° C. The ethylene glycol may be heated to a temperature of up to 230° C., preferably up to 220° C., and more preferably up to 210° C. Thus, the ethylene glycol may be heated to a temperature of from 150 to 230° C., preferably from 170 to 220° C., and more preferably from 190 to 210° C. The catalyst system is used in step (a) to improve the depolymerisation reaction. The catalyst system preferably comprises a transition metal catalyst, such as a zinc-containing catalyst. Suitable zinc catalysts include zinc acetate.

In some embodiments, the catalyst system consists of a transition metal catalyst. However, in preferred embodiments, the catalyst system comprises a catalyst, e.g. as described above, in a carrier. Suitable carriers include nitrogen-containing carriers, such as urea.

Urea has surprisingly been found to be highly effective at maintaining metals (e.g. the transition metal catalyst component of the catalyst system; or traces of metal catalysts that were used to produce the PET originally, such as antimony catalysts) and other contaminants in solution, thereby enabling these components to be separated from BHET in step (b). The urea may also be used to solubilise contaminants in the PET recycling process. It has surprisingly been found that a eutectic salt catalyst system is particularly effective at solubilising metals and/or contaminants.

The carrier may be used in the catalyst system in an amount of at least 1 times, preferably at least 2 times, and more preferably at least 3 times the molar quantity of transition metal cation in the transition metal catalyst. The carrier may be used in an amount of up to 8 times, preferably up to 6 times, and more preferably up to 5 times the molar quantity of transition metal cation. Thus, the carrier may be used in an amount of from 1 to 8 times, preferably from 2 to 6 times, and more preferably from 3 to 5 times the molar quantity of transition metal cation. These ratios of carrier to transition metal catalyst have been found to give high rates of reaction, whilst retaining metal ions in solution. As mentioned above, the transition metal cation will typically be a zinc cation.

Most preferred for use in step (a) are catalyst systems comprising, and preferably consisting of, zinc acetate and urea, and in particular a catalyst system having the formula [nNH₂CONH₂·ZnOAc], where n is from 1 to 7, for instance n may be 3, 4 or 5. This catalyst system advantageously forms a eutectic salt.

The catalyst system may be in the liquid phase during step (a), and preferably throughout the PET recycling method.

The catalyst system may be used in step (a) in an amount of at least 0.001 times, preferably at least 0.003 times, and more preferably at least 0.004 times the amount of PET by weight. The catalyst system may be used in step (a) in an amount of up to 1 times, preferably up to 0.01 times, and more preferably up to 0.006 times the amount of PET by weight. Thus, the catalyst system may be used in step (a) in an amount of from 0.001 to 1 times, preferably from 0.003 to 0.01 times, and more preferably from 0.004 to 0.006 times the amount of PET by weight.

At least 60%, preferably at least 80%, and more preferably at least 95% by weight of the catalyst system may be added to the first reactor. However, as mentioned above, all of the catalyst system is preferably added to the first reactor. It will be appreciated that, where less than 100% of the catalyst system is added to the first reactor, the remainder is added to the series of depolymerisation reactors downstream of the first depolymerisation reactor.

Step (a) is generally carried out in the absence of any solvents beyond ethylene glycol and any carriers that may be present in the catalyst system. It will be appreciated that there may be some residual liquid, e.g. water, that has been passed to the claimed process as a coating on the PET due to washing; however, this is not considered to be a solvent for the purposes of the present invention. Thus, solvent may be present in step (a) in an amount of up to 0.1 times, preferably up to 0.01 times, and more preferably up to 0.001 times the amount of PET used in step (a) by weight. Most preferably, substantially no solvent is present in step (a).

Preferably, water is removed from the depolymerised mixture between steps (a) and (b), such as in a moisture evaporation vessel. For instance, water may be flashed from the depolymerised mixture and therefore the moisture evaporation vessel may be a flash tank. A moisture separator may be installed in the vacuum line to condense the water. Some ethylene glycol may be flashed off at the same time as the water in the form of a water-ethylene glycol azeotrope.

Water may be removed from the depolymerised mixture at a temperature of at least 150° C., preferably at least 170° C., and more preferably at least 190° C. Water may be removed from the depolymerised mixture at a temperature of up to 230° C., preferably up to 220° C., and more preferably up to 210° C. Thus, water may be removed from the depolymerised mixture at a temperature of from 150 to 230° C., preferably from 170 to 220° C., and more preferably from 190 to 210° C.

Preferably, water is removed from the depolymerised mixture under vacuum. Water may be removed from the depolymerised mixture at a pressure of at least 50 kPa, preferably at least 65 kPa, and more preferably at least 75 kPa. Water may be removed from the depolymerised mixture at a pressure of up to 100 kPa, preferably up to 90 kPa, and more preferably up to 85 kPa. Thus, water may be removed from the depolymerised mixture at a pressure of from 50 to 100 kPa, preferably from 65 to 90 kPa, and more preferably from 75 to 85 kPa.

Water may be removed until a water content, by weight, of 0.5% or less, preferably 0.3% or less, and more preferably 0.1% or less, is reached in the depolymerised mixture. This means that the depolymerised mixture passed to step (b) is substantially free from water.

The water that is removed from the depolymerised mixture between steps (a) and (b) may be recycled to step (c) for use as the protic solvent.

Preferably, the depolymerised mixture is separated from any insoluble components between steps (a) and (b). Insoluble components include unreacted PET (though the levels of this will typically be very low, if present at all) and other inert solids. Other solids may include non-PET polymers such as polyethylene (PE) and polypropylene (PP). Preferably, insoluble components are removed from the depolymerised mixture by centrifugation, for example using a centrifugal separator. The centrifugal separator may comprise a centrifugal drum in which a plurality of plates, preferably curved plates, are disposed so as to form channels in the centrifugal drum. Such centrifugal filters include Evodos® centrifugal separators. Preferably, two centrifugal separators are used which operate in tandem to provide continuous flow. A storage tank may further be provided downstream of the centrifugal separators to aid in flow continuity to the downstream process.

Alternatively, other techniques may also be used such as passing the depolymerised mixture through a filter to remove insoluble components. Tricanters may be used in order to achieve very high levels of solid-liquid separation.

The depolymerised mixture may be cooled before it is separated from any insoluble components between steps (a) and (b). This is to encourage the precipitation of unconverted materials. The depolymerised mixture may be cooled to a temperature of up to 150° C., preferably up to 130° C., and more preferably up to 110° C. The depolymerised mixture may be cooled to a temperature of at least 80° C., preferably at least 90° C., and more preferably at least 95° C. Thus, the depolymerised mixture may be cooled to a temperature of from 80 to 150° C., preferably from 90 to 130° C., and more preferably from 95 to 110° C.

Where water and insoluble components are removed from the depolymerised mixture between steps (a) and (b), water is preferably removed before the insoluble components.

Preferably, the depolymerised mixture is heated before being fed to the evaporator for evaporation crystallisation in step (b). Pre-heating of the depolymerised mixture may be performed in a heat exchanger such as a steam-fed shell-and-tube heat exchanger which preferably uses steam as the heating medium. The depolymerised mixture may be heated to a temperature of at least 150° C., preferably at least 170° C., and more preferably at least 190° C. The depolymerised mixture may be heated to a temperature of up to 250° C., preferably up to 230° C., and more preferably up to 210° C. Thus, the depolymerised mixture may be heated to a temperature of from 150 to 250° C., preferably from 170 to 230° C., and more preferably from 190 to 210° C.

In step (b) of the method, a precipitate comprising BHET is crystallised by removing a volatiles stream comprising ethylene glycol from the depolymerised mixture formed in step (a) using evaporation crystallisation. Evaporation crystallisation is a process by which a material is concentrated precipitated by, at least in part, removing solvent. A variety of evaporators may be used for carrying out step (b), with wiped film evaporators particularly preferred. Wiped film evaporators advantageously remove a high proportion of the ethylene glycol, and encourage a high yield of BHET product. In other crystallisation techniques, BHET product may be left behind in solution. Though evaporation crystallisation is preferred, it is also envisaged that other crystallisation methods may be used in step (b) such as cooling crystallisation.

Step (b) may be carried out at a temperature of at least 150° C., preferably at least 170° C., and more preferably at least 190° C. Step (b) may be carried out at a temperature of up to 250° C., preferably up to 230° C., and more preferably up to 210° C. Thus, step (b) may be carried out at a temperature of from 150 to 250° C., preferably from 170 to 230° C., and more preferably from 190 to 210° C. At these temperatures, the precipitate comprising BHET may be partially or fully in the form of a melt.

Step (b) is generally carried out under vacuum. Step (b) may be carried out at a pressure of up to 50 kPa, preferably up to 30 kPa, and more preferably up to 15 kPa. Step (b) may be carried out at a pressure of at least 0.1 kPa, preferably at least 1 kPa, more preferably at least 5 kPa. Thus, step (b) may be carried out at a pressure of from 0.1 to 50 kPa, preferably from 1 to 30 kPa, and more preferably from 5 to 15 kPa.

Typically steps (a) and (b) will be carried out at similar temperatures (e.g. within 30° C., preferably within 20° C., and more preferably within 10° C. of one another), but with a lower pressure used in step (b) than step (a) (e.g. at least 50 kPa, preferably at least 70 kPa, and more preferably at least 80 kPa lower).

Preferably, the majority of the ethylene glycol that is present in the depolymerised mixture formed in step (a) is removed as part of the volatiles stream in step (b). Thus, the volatiles stream in step (b) may comprise at least 70% by weight, preferably at least 80% by weight, and more preferably at least 90% by weight of the ethylene glycol present in the depolymerised mixture formed in step (a). By removing a high proportion of ethylene glycol with the volatiles stream, any subsequent separation of ethylene glycol and the protic solvent that is added in step (c) is less energy intensive.

It is not necessary to remove all of the ethylene glycol in step (b), with at least 5% by weight of the ethylene glycol that is present in the depolymerised mixture typically remaining with the precipitate comprising BHET at the end of step (b).

The evaporated volatiles stream produced in step (b) may be condensed using a condenser.

Preferably, the ethylene glycol that is removed in step (b) as part of the evaporated volatiles stream is recycled to the series of depolymerisation reactors in step (a). The ethylene glycol may be separated from other components that may be present in the volatiles stream before recycling. In some embodiments, the recycled ethylene glycol stream comprises less than 2%, preferably less than 1%, and more preferably less than 0.5% by weight of components other than ethylene glycol.

Step (b) may be carried out for a period of at least 10 minutes, preferably at least 20 minutes, and more preferably at least 25 minutes. Step (b) may be carried out for a period of up to 120 minutes, preferably up to 45 minutes, and more preferably up to 35 minutes. Thus, step (b) may be carried out for a period of from 10 to 120 minutes, preferably from 20 to 45 minutes, and more preferably from 25 to 35 minutes.

The depolymerised mixture may be stirred during step (b), though this is not necessary.

The conditions used in step (a) may lead to a precipitate containing a high proportion of BHET. BHET may be present in the precipitate in an amount of at least 95%, preferably at least 99%, and more preferably at least 99.5% by weight.

The precipitate formed in step (b) comprises BHET but will typically also comprise dimers and trimers of BHET, e.g. in an amount of at least 0.01% by weight. Dimers and trimers of BHET may be present in the precipitate in an amount of up to 2%, preferably up to 0.5%, and more preferably up to 0.2% by weight. The amount of different components in the precipitate formed in step (b) may be determined using standard techniques, such as high performance liquid chromatography (HPLC). HPLC may be carried out using the following conditions—instrument: Shimazu LC-20A HPLC; detector: photo-diode array (PDA) detector, chromatogram centre wavelength of 223 nm (4 nm ‘slit’ bandwidth); column: C18; mobile phase: 30% water 70% methanol; flow rate: 0.5 ml/min; oven temp: 35° C.; sample: dissolved in methanol; injection volume: 20 uL. Samples are quantified by external standard method.

In step (c) of the method, the precipitate formed in step (b) is dissolved in a protic solvent to form a solution comprising BHET.

A wide range of protic solvents may be used in step (c). For instance, the protic solvent may be selected from water and alcohols. Preferably, the protic solvent is selected from water and C1 to C12 alcohols such as methanol, ethanol, propanol (e.g. iso-propanol), and butanol (e.g. n-butanol or tert-butanol). More preferably, the protic solvent is selected from water and methanol, and most preferably the protic solvent is water.

While the use of protic solvent is particularly preferred in step (c), in some instances, the solvent used in step (c) may be instead an aprotic solvent. For instance, the solvent used in step (c) may be an ether or ester, preferably selected from dimethyl carbonate (DMC), dimethoxyethane (DME) or diisopropylether (DIPE).

Mixtures of any of the aforementioned solvents may also be used in step (c).

Preferably, water is used as the protic solvent in step (c). Dimers and trimers of BHET are insoluble in water and thus, in step (c), the BHET dissolves to form an aqueous phase, while the dimers and trimers remain as solid materials which can be separated from the aqueous phase, e.g. by filtration, at the end of step (c). The aqueous solution can then be recrystallised in step (e), with the purified product used as a high quality monomer feedstock.

Alternatively, in step (c) of the method, the precipitate formed in step (b) may be dissolved in methanol to form a solution comprising BHET. It has surprisingly been found that methanol is an excellent solvent for use in step (c), as it provides high levels of decolouration of the precipitate formed in step (b) as well as low levels of product loss. However, the use of water is preferred as dimers and trimers of BHET are partially soluble in methanol and hence these are retained in detectable quantities in the monomer product if methanol is used for the recrystallisation in step (c) of the method.

Step (c) may be carried out at a temperature of at least 60° C., preferably at least 80° C., and more preferably at least 90° C. Step (c) may be carried out at a temperature of up to 100° C., preferably up to 98° C., and more preferably up to 95° C. Thus, step (c) may be carried out at a temperature of from 60 to 100° C., preferably from 80 to 98° C., and more preferably from 90 to 95° C.

Preferably, the solvent used in step (c) is heated prior to being added to the precipitate formed in step (b), for example before entering the dissolution vessel. Pre-heating of the solvent may be performed in a heat exchanger, for example a shell-and-tube heat exchanger. Preferably, the heat exchanger uses heated water or steam from the outlet of the moisture evaporation vessel as the heating medium. It will be appreciated that the temperature that the solvent is heated to depends upon the solvent used, in particular the boiling point of the solvent. Preferably the solvent is not boiling. When water is used as the protic solvent in step (c) the temperature is preferably less than 100° C., and when methanol is used the temperature is preferably less than 64° C. Preferably, the temperature of the solvent is at least 55° C.

Step (c) may be carried out at atmospheric pressure, i.e. without the application or removal of pressure.

Step (c) may be carried out fora period of at least 5 minutes, preferably at least 10 minutes, and more preferably at least 20 minutes. Step (c) may be carried out for a period of up to 60 minutes, preferably up to 50 minutes, and more preferably up to 40 minutes. Thus, step (c) may be carried out for a period of from 5 to 60 minutes, preferably from 10 to 50 minutes, and more preferably from 20 to 40 minutes.

Dissolution of the precipitate may be carried out with stirring, though this is not necessary.

The protic solvent, e.g. water, may be used in step (c) in an amount of at least 0.1 times, preferably at least 0.12 times, and more preferably at least 0.15 times the amount of PET used in step (a) by weight. Water may be used in step (c) in an amount up to 1 times, more preferably up to 0.5 times, and more preferably up to 0.25 times the amount of PET used in step (a) by weight. Thus, water may be used in step (c) in an amount of from 0.1 to 1 times, preferably from 0.12 to 0.5 times, and most preferably from 0.15 to 0.25 times the amount of PET used in step (a) by weight.

Though less preferred, when methanol alone is used as the solvent in step (c), it may be used in an amount of at least 1 times, preferably at least 1.5 times, and more preferably at least 2 times the amount of PET used in step (a) by weight. Methanol may be used in step (c) in an amount of up to 10 times, preferably up to 5 times, and more preferably up to 3 times the amount of PET used in step (a) by weight. Thus, methanol may be used in step (c) in an amount of from 1 to 10 times, preferably from 1.5 to 5 times, and more preferably from 2 to 3 times the amount of PET used in step (a) by weight.

In step (d) of the method, impurities are removed from the solution produced in step (c) to give a purified solution comprising BHET. Preferably, step (d) comprises decolourising the solution. This may be done by contacting the solution with one or more decolourising agents. Step (d) may also comprise removing other contaminants such as metals and catalyst residues from the solution produced in step (c).

Preferably, step (d) is carried out by passing the solution produced in step (c) through an exchange bed, and most preferably a plurality of exchange beds in series, packed with one or more purifying (e.g. decolourising) agents. For example, each exchange bed in series may be packed with a different purifying agent.

The one or more purifying agents used in step (d) may include carbon (e.g. activated carbon, preferably having a high pore volume and surface area), a resin, such as an ion exchange resin, preferably a cation exchange resin, such as an acidic cation exchange resin, preferably comprising sulfonic acid or carboxylic acid groups, with sulfonic acid groups preferred, or alternatively or in addition an anion exchange resin, such as a basic anion exchange resin, preferably comprising quaternary ammonium salts, and/or a clay (e.g. activated clays such as bentonite and montmorillonite clays). Preferably, the solution produced in step (c) is contacted with carbon and an exchange resin.

In particularly preferred embodiments of the method, the solution produced in step (c) is contacted with a plurality of different purifying agents via passage through a plurality of exchange beds arranged in series. For example, a first exchange bed may comprise an activated carbon (e.g. as a decolourising agent), a second exchange bed may comprise an exchange resin which is preferably an organic scavenger bed (e.g. for removing hydrophobic organic species), and a third exchange bed may comprise a cation exchange resin. The first to third exchange beds may be arranged in series so that the solution produced in step (c) passes through each in step (d).

The solution produced in step (c) may be passed through one or more exchange beds of each type. Preferably, the solution produced in step (c) is passed through at least two, and preferably two, exchange beds of each type. Therefore, the solution produced in step (c) is preferably passed through two of the first exchange beds, two of the second exchange beds and two of the third exchange beds described above.

The one or more exchange beds that may be used in step (d) may be periodically regenerated. Preferably each of the exchange beds is periodically regenerated. The exchange beds may be regenerated using steam, an acidic solution or a basic solution. The exchange beds may also be regenerated using a gas, e.g. nitrogen or hydrogen, preferably at elevated temperature. Preferably, activated carbon beds and cation exchange beds are regenerated with steam. Organic scavenger beds may be regenerated with an acidic solution. Other known methods of regeneration may also be used.

During regeneration of an exchange bed, a reserve exchange bed of the same type is used for purifying the solution. This means that the process need not be halted during regeneration of the exchange bed.

Step (d) may be carried out at a temperature of at least 40° C., preferably at least 55° C., and more preferably at least 70° C. Step (d) may be carried out at a temperature of up to 110° C., preferably up to 100° C., and more preferably up to 90° C. Thus, step (d) may be carried out at a temperature of from 40 to 110° C., preferably from 55 to 100° C., and more preferably from 70 to 90° C.

Step (d) may be carried out at atmospheric pressure, i.e. without the application or removal of pressure.

Step (d) may be carried out for a period of at least 10 minutes, preferably at least 25 minutes, and more preferably at least 40 minutes. Step (d) may be carried out for a period of up to 120 minutes, preferably up to 100 minutes, and more preferably up to 60 minutes. Thus, step (d) may be carried out for a period of from 10 to 120 minutes, preferably from 25 to 100 minutes, and more preferably from 40 to 80 minutes.

Though less preferred, in some embodiments purification step (d) may be omitted. This is because the purification provided as a result of recrystallisation, for example in methanol, alone may be sufficient for producing a decoloured purified product comprising BHET, though typically such products will be used in low grade applications such as carpets. Thus, in some embodiments, a purified product comprising BHET may be crystallised in step (e) from the solution produced in step (c).

One of the advantages of using methanol in step (c) of the PET recycling method is that the solution may be formed in step (c), purified in step (d) and passed to step (e) for crystallisation without being filtered. This is because methanol dissolves BHET and, unlike water, also dimers and trimers of BHET. While carrying the dimers and trimers through a PET recycling process may be avoided by filtering them out of an aqueous system, step (a) of the PET recycling method produces dimers and trimers in such low amounts that they may be carried through the recycling process with BHET. Thus, in some embodiments, a solid-liquid separation step is not carried out between steps (c) and (e) of the PET recycling method.

However, when water is used in step (c) of the PET recycling method, it is advantageous to remove solid components from the BHET solution between steps (c) and (d), to remove BHET dimers and trimers, which are insoluble in water. It is also preferable to remove solid components from the solution comprising BHET between steps (c) and (d) when solvents other than water or methanol are used.

Solid components that may be found in the solution comprising BHET that is formed in step (c) include oligomers of BHET, such as dimers and trimers of BHET. Once separated from the solution comprising BHET, the oligomers of BHET are preferably recycled to the depolymerisation reactors in step (a), preferably the first depolymerisation reactor.

Other solid components that may be found in the BHET solution include IPA. IPA is particularly insoluble in water and this is one of the reasons that water is preferably used as the protic solvent in step (c). Therefore, IPA is preferably removed from the solution comprising BHET upon removal of the insoluble components.

Where the solid components comprise IPA, the IPA is preferably recovered from other solid components. In particular, the IPA is preferably separated from the oligomers of BHET before they are recycled to the depolymerisation reactors in step (a). Separation of IPA from the oligomers of BHET may be carried out using chromatography, for instance in a simulated moving bed process, or using selective solvent dissolution.

Solid components may be removed from the solution comprising BHET by centrifugation, for example using a centrifugal separator. The centrifugal separator preferably comprises a centrifugal drum in which a plurality of plates, preferably curved plates, are disposed so as to form channels in the centrifugal drum. Such centrifugal filters include Evodos® centrifugal separators. Preferably, two centrifugal separators are used which operate in tandem to provide continuous flow. A storage tank may further be provided downstream of the centrifugal separators to aid in flow continuity to the downstream process.

Other solid separation techniques may also be used, such as passing the solution comprising BHET through a filter to remove insoluble components. Tricanters may be used in order to achieve very high levels of solid-liquid separation.

In step (e) of the method, a purified product comprising BHET is crystallised from the purified solution.

Step (e) is preferably carried out using cooling crystallisation. Suitable crystallisers include stirred or wall-scraped crystallisers. The purified solution produced in step (d) may be left to cool naturally, though it is preferably it is cooled using a coolant. The coolant may be present in a jacket which surround the crystalliser, or it may be passed through a series of heat exchangers through which the purified solution is also passed, e.g. in countercurrent flow.

Especially when the solvent used in step (c) is water, step (e) may be carried out by reducing the temperature of the purified solution to a temperature of at least 0° C., preferably at least 10° C., and more preferably at least 20° C. Step (e) may be carried out by reducing the temperature of the purified solution to a temperature of up to 55° C., preferably up to 45° C., and more preferably up to 40° C. Thus, step (e) may be carried out by reducing the temperature of the purified solution to a temperature of from 0 to 55° C., preferably 10 to 45° C., and more preferably 20 to 40° C.

Especially when the solvent used in step (c) is methanol, step (e) may be carried out by reducing the temperature of the purified solution to a temperature of at least 0° C., preferably at least 5° C., and more preferably at least 8° C. Step (e) may be carried out by reducing the temperature of the purified solution to a temperature of up to 30° C., preferably up to 15° C., and more preferably up to 10° C. Thus, step (e) may be carried out by reducing the temperature of the purified solution to a temperature of from 0 to 30° C., preferably from 5 to 15° C., and more preferably from 8 to 12° C.

Step (e) may be carried out at atmospheric pressure, i.e. without the application or removal of pressure. Step (e) may also be carried out under vacuum, and this is preferred when melt crystallisation is used (discussed below).

Step (e) may be carried out for a period of at least 10 minutes, preferably at least 20 minutes, and more preferably at least 25 minutes. Step (e) may be carried out for a period of up to 60 minutes, preferably up to 45 minutes, and more preferably up to 35 minutes. Thus, step (e) may be carried out for a period of from 10 to 60 minutes, preferably from 20 to 45 minutes, and more preferably from 25 to 35 minutes.

The purified solution may be stirred during step (e).

The purified product that is formed in step (e) may contain a high proportion of BHET. BHET may be present in the purified product in an amount of at least 95%, preferably at least 99%, and more preferably at least 99.5% by weight.

If methanol is used as solvent in step (c), the purified product formed in step (e) may also comprise dimers and trimers of BHET, e.g. in an amount of at least 0.01% by weight. Dimers and trimers of BHET may be present in the purified product in an amount of up to 2%, preferably up to 0.5%, and more preferably up to 0.2% by weight. Preferably, amounts of dimers and trimers that are present in the purified product formed in step (e) are substantially the same as the amounts of dimers and trimers that are present in the precipitate formed in step (b).

The amounts of different components in the purified product formed in step (e) may be determined using the methods described above.

Preferably, IPA is present in the purified BHET product formed in step (e) in an amount of up to 0.5%, preferably up to 0.2%, and more preferably up to 0.1% by weight. A high proportion of the IPA that is present in the PET feed is removed during the recycling process. Thus, amount of IPA (% by weight) in the purified BHET product formed in step (e) may be up to 20%, preferably up to 10%, and more preferably up to 5% of the amount of IPA (% by weight) that is present in the PET that is depolymerised in step (a).

The amount of IPA in the purified BHET product may be determined using standard techniques, such as NMR. NMR may be carried out using the following conditions—spectra were acquired in d₂-tetrachloroethane solvent (Goss Scientific D, 99.8%) at ambient laboratory temperature and auto referenced against the solvent peak using a JEOL ECS 400 NMR spectrometer. The NMR is preferably proton NMR.

A key advantage of the PET recycling method is that it may be used to produce purified products having low b[h] values, in particular b[h] values of 2 or less. PET prepared from BHET having these colour densities is of a very high grade, and may be used in applications which require excellent visual appearance such as in transparent and colour-free water bottles. Thus, the purified product that is formed in step (e) may exhibit a b[h] value of up to 2, e.g. from 0 to 2. In some instances, the purified product may be used in lower grade applications, e.g. in carpets or films, in which case it may have a b[h] value of up to 4, for instance up to 3.

The PET recycling method may be used to form a purified product in step (e) with a b[h] value that is 0.5 times, preferably 0.1 times, and more preferably 0.05 times that of the PET that is used in step (a). By using preferred embodiments of the PET recycling method, even higher reductions in b[h] value are obtainable, for instance where the PET feed used in step (a) exhibits a high colour density.

Colour density of the purified product that is formed in step (e) may be measured as described above in connection with the PET that is used in step (a).

The purified product comprising BHET is preferably separated from the protic solvent (and preferably other liquid components such as ethylene glycol) after step (e) and, where drying step (f) is present, before step (f). The precipitate may be isolated using known methods, e.g. by filtration or centrifugation. Preferably the purified BHET product is isolated using a filter press.

It will be appreciated that the liquid that remains after crystallising in step (e), and thus the residual liquid that remains after isolation of the purified BHET product, will comprise the protic solvent and ethylene glycol. The ethylene glycol will typically be present in just small amounts, since it is preferably mostly separated from the BHET precipitate that is formed in step (b). The protic solvent is preferably recycled for use in step (c). The protic solvent may be recycled to step (c) with the residual liquid that remains after isolation of the purified BHET product or, as discussed in more detail below, it may be isolated from the residual liquid before being recycled to step (c).

In some instances, the PET recycling method may further comprise isolating ethylene glycol from the residual liquid that remains after isolation of the purified BHET product. For example, ethylene glycol may be separated from the residual liquid which comprises the protic solvent using low pressure evaporation and condensation. The ethylene glycol may be recycled for use in step (a), and more preferably to the first depolymerisation reactor.

One of the principal advantages of using methanol to carry out step (c), rather than water, is that methanol and ethylene glycol may be readily recovered. Thus, the recovery of methanol and ethylene glycol from the residual liquid may be carried out in a single stage evaporator. In contrast, when water is used, recovery of ethylene glycol and water from the residual liquid can be challenging, since water and ethylene glycol form an azeotropic mixture. Thus, where water is used in step (c), the use of a multi-stage evaporator is preferred for recovering water and ethylene glycol from the residual liquid. However, if the majority of ethylene glycol is removed in step (b), as described hereinabove, the recovery of water from a mixture of ethylene glycol may not be required.

When methanol is used in step (c), the recovery of methanol and ethylene glycol from the residual liquid may be carried out by heating the residual liquid to a temperature between the boiling points of methanol and ethylene glycol. For instance, the residual liquid may be heated to a temperature of greater than 65° C., preferably greater than 70° C. and more preferably greater than 75° C. The residual liquid may be heated to a temperature of up to 120° C., preferably up to 100° C., and more preferably up to 90° C. Thus, the residual liquor may be heated to a temperature of from 65 to 120° C., 70 to 100° C., and more preferably from 70 to 90° C.

The recovery of methanol and ethylene glycol from the residual liquid may be carried out at ambient pressure, i.e. without the application or removal of pressure.

Typically, the residual liquid will not be further processed before it is processed to recover methanol and ethylene glycol. Preferably, the methanol is not further processed before being recycled for use in step (c).

When water is used in step (c), a two stage evaporator process is preferred to recover water and ethylene glycol. In a first evaporator, water may be recovered from the residual liquid by application of low pressure, allowing evaporation at reduced temperature; for example, operation of the evaporator at a pressure at or about 10 kPa is preferred, with associated condenser temperature at or about 46° C. and reboiler temperature at or about 132° C. The residual ethylene glycol can then be recovered in a second evaporator by application of low pressure, operating preferably at a pressure at or about 0.08 bar, and a temperature at or about 138° C. The skilled person will appreciate that other operating temperatures and pressures may also be selected for the first and second evaporators. Enhanced recovery of water may be achieved if desired through operating the first evaporator at lower temperature, or by the use of molecular sieves downstream of the first evaporator. Preferably, the evaporators are distillation columns.

Ethylene glycol may, however, be subject to further purification before it is recycled to step (a). For instance, ethylene glycol may be flashed to separate any organic waste that is entrained therein.

Flashing may take place at a temperature of at least 130° C., preferably at least 150° C., and more preferably at least 170° C. Flashing may take place at a temperature of up to 230° C., preferably up to 210° C., and more preferably up to 190° C. Thus, flashing may take place at a temperature of from 130 to 230° C., preferably from 150 to 210° C., and more preferably from 170 to 190° C.

Flashing typically takes place under reduced pressure. For instance, flashing may take place at a pressure of up to 80,000 Pa, preferably up to 60,000 Pa, and more preferably up to 40,000 Pa. Flashing may take place at a pressure of at least 10,000 Pa, preferably at least 15,000 Pa, and more preferably at least 20,000 Pa. Thus, flashing may take place at a pressure of from 10,000 to 80,000 Pa, preferably from 15,000 to 60,000 Pa, and more preferably from 20,000 to 40,000 Pa.

When methanol is used in step (c), the recovery of methanol is so effective (even at industrial scales such as those described herein) that, when the recovered methanol is recycled to step (c), non-recycled methanol need only be added in step (c) in an amount of up to 0.008 times, preferably up to 0.006 times, and more preferably up to 0.005 times the amount of PET used in step (a) by weight. Non-recycled methanol may be used in step (c) an amount of at least 0.001 times, preferably at least 0.003 times, and more preferably at least 0.004 times the amount of PET used in step (a) by weight. Thus, non-recycled methanol may be used in step (c) in an amount of from 0.001 to 0.008 times, preferably from 0.003 to 0.006 times, and more preferably from 0.004 to 0.005 times the amount of PET used in step (a) by weight. Thus, it will be appreciated that the amount of methanol that is lost during the PET recycling method is extremely low, and much lower than the amount of water that would be lost when used in place of methanol in step (c).

However, when water is used as the solvent in step (c), it may also be effectively recovered so that at least a majority of the water used in step (c) is recycled, preferably using the two stage evaporator process described hereinabove. The water lost is typically removed from the system as humid air. Given the minimal environmental impact of water loss from the system, compared to methanol-containing waste, and the energy cost associated with water recovery, it may not be beneficial to maximize water recycling.

The PET recycling method may further comprise step (f), in which the purified product comprising BHET is dried. Drying is preferably performed in the crystallisation system in which BHET is crystallised from the purified solution in step (e). Where melt crystallisation is used in step (e) (discussed below), the purified product comprising BHET is dried as part of the melt crystallisation process.

The product may be dried by passing air over the purified product, e.g. in a fluidised bed drier. Drying may also take place in a belt dryer or in a rotary dryer (e.g. a rotary vacuum dryer). Where a filter is used to separate the purified BHET precipitate from the liquid that remains after crystallising step (e), drying may be carried out by air drying the filter cake.

The air may be heated to a temperature of at least 30° C., preferably at least 40° C., and more preferably at least 50° C. The air may be heated to a temperature of up to 100° C., preferably up to 90° C., and more preferably up to 80° C. Thus, the air may be heated to a temperature of from 30 to 100° C., preferably from 40 to 90° C., and more preferably from 50 to 80° C.

Drying step (f) may be carried out at ambient pressure, i.e. without the application or removal of pressure, though, where a rotary vacuum dryer is used, the drying step will be carried out under vacuum.

Drying step (f) may be conducted for a period of at least 10 minutes, preferably at least 15 minutes, and more preferably at least 20 minutes. Drying step (f) may be carried out for a period of up to 60 minutes, preferably up to 50 minutes, and more preferably up to 40 minutes. Thus, drying step (f) may be carried out for a period of from 10 to 60 minutes, preferably from 15 to 50 minutes, and more preferably from 20 to 40 minutes.

In preferred embodiments, step (e) of the method is carried out using melt crystallisation. Thus, step (e) may be carried out in a melt crystalliser. In these embodiments, a purified product comprising BHET may be crystallised from the purified solution (e.g. using the cooling crystallisation described above), isolated (e.g. as described above), dried (e.g. as described above) and melted. A melter is used for melting the purified BHET product. The use of melt crystallisation in step (e) promotes the formation of relatively large and pure BHET crystals, thereby enabling a high proportion of the BHET to be recovered from the liquid that remains after crystallising in step (e).

The purified BHET product may be melted at a temperature of at least 106° C., preferably at least 108° C. and more preferably at least 110° C. The purified BHET product may be melted at a temperature of from up to 150° C., preferably up to 130° C., and more preferably up to 120° C. Thus, the purified BHET product may be melted at a temperature of from 106 to 150° C., preferably from 108 to 130° C., and more preferably from 110 to 120° C. The present inventors have found that BHET melts are surprisingly unstable, and these temperatures have been found to prevent instability without compromising on the flowability of the melt.

The PET recycling method may be operated in a batch mode or a continuous mode, though it is preferably operated continuously.

The PET recycling method is preferably carried out on an industrial scale. Thus, the method may recycle at least 10 tonne/day, preferably at least 30 tonne/day, and potentially at least 100 tonne/day of PET.

A key advantage of the present invention is that the recycled BHET product may be used directly in the polymerisation reaction, i.e. it is not subjected to further purification before use. In particular, because the recycled BHET product of the present invention comprises low amounts of IPA, the amount of IPA present in the recycled BHET product need not be measured before, or during, polymerisation. This simplifies the polymerisation reaction compared to typical prior art methods.

Thus, the amount of IPA present in the purified BHET is preferably not measured prior to carrying out the polymerisation reaction of the present invention. In particular, the amount of IPA is preferably not measured in the purified BHET product, or during the production of the purified BHET product. The amount of IPA that is present during the polymerisation reaction is preferably also not measured.

The method of the present invention provides a polymer comprising constitutional units derived from BHET, i.e. a PET polymer. In some embodiments, the polymer is a PET homopolymer. A PET homopolymer is substantially free from constitutional units other than those derived from BHET.

However, generally the polymer that is prepared using the method of the present invention is a PET copolymer. In contrast with a homopolymer, a PET copolymer comprises constitutional units other than those derived from BHET.

The PET copolymer may be prepared from a monomer mixture containing the recycled BHET product in an amount of at least 25%, preferably at least 50%, and more preferably at least 90% by weight of monomers. The PET copolymer may be prepared from a monomer mixture containing the recycled BHET product in an amount of up to 99.5%, preferably up to 99%, and more preferably up to 97% by weight of monomers. Thus, the PET copolymer may be prepared from a monomer mixture containing the recycled BHET product in an amount of from 25 to 99.5%, preferably from 50 to 99%, and more preferably from 90 to 97% by weight of monomers.

The PET copolymer may comprise a wide variety of constitutional units other than those derived from BHET. For instance, the PET copolymer may comprise constitutional units derived from IPA, diethylene glycol (DEG), butanediol (e.g. 1,4-butanediol), propanediol (e.g. 1,3-propanediol) or cyclohexanedimethanol (CHDM). Thus, the polymer may be prepared from a monomer mixture which contains IPA, diethylene glycol (DEG) or cyclohexanedimethanol (CHDM). Combinations of these constitutional units/monomers may also be used. Which monomers are used, and the amounts in which they are used, will depend on the desired properties of the PET copolymer.

In preferred embodiments, the PET copolymer comprises constitutional units derived from IPA. The PET copolymer may be prepared from a monomer mixture containing IPA in an amount of at least 0.5%, preferably at least 0.8%, and more preferably at least 1% by weight of monomers. The PET copolymer may be prepared from a monomer mixture containing IPA in an amount of up to 30%, preferably up to 20%, and more preferably up to 10% by weight of monomers. Thus, the PET copolymer may be prepared from a monomer mixture containing IPA in an amount of from 0.5 to 30%, preferably from 0.8 to 20%, and more preferably from 1 to 10% by weight of monomers.

Where the monomer mixture comprises IPA, in some embodiments, the method comprises adding IPA to the monomer mixture in a form in which other monomers are not added along with the IPA, i.e. in an isolated form. In other embodiments, the IPA may be added in the form of a BHET product which comprises IPA, i.e. in the form of “dirty” BHET such as BHET derived from PET using conventional methods.

In preferred embodiments, the recycled BHET product may be blended with another BHET source. Thus, the polymerisation method may comprise:
blending the recycled BHET product with a second BHET product to form a blended BHET stream; and
carrying out a polymerisation reaction on the blended BHET stream.

The second BHET product is preferably a recycled BHET product. The second BHET product may be prepared using conventional methods and, as such, preferably comprises IPA in an amount of at least 0.5%, preferably at least 0.8%, and more preferably at least 1% by weight. Thus, the recycled BHET product which contains minimal amounts of IPA may be used to clean-up the second “dirty” BHET product. In some instances, the second BHET product comprises IPA in an amount of at least 10% by weight.

The first and second BHET products may be blended in proportions which give a target % by weight IPA in the blended stream. The first BHET product is assumed to be entirely free from IPA for this purpose. Thus, the method may comprise blending the first and second BHET products in the weight ratio F:S, wherein:

F=1−S

S=% IPA_{Target}/% IPA_{SecondBHET}
where: F is the first BHET product;
   S is the second BHET product;
   % IPA_{Target} represents the target % by weight of IPA in the blended stream; and
   % IPA_{SecondBHET} represents the % by weight of IPA in the second BHET product.

Suitable conditions for preparing PET are well known in the art, and such conditions may be used to polymerise the recycled BHET product described herein.

The polymerisation reaction may be carried out at a temperature of at least 200° C., preferably at least 230° C., and more preferably at least 250° C. The polymerisation reaction may be carried out at a temperature of up to 350° C., preferably up to 320° C., and more preferably up to 300° C. Thus, the polymerisation reaction may be carried out at a temperature of from 200 to 350° C., preferably from 230 to 320° C., and more preferably from 250 to 300° C.

The polymerisation reaction may be carried out under vacuum. For instance, the polymerisation reaction may be carried out at a pressure of up to 80 kPa, preferably up to 10 kPa, and more preferably up to 1.0 kPa.

The polymerisation reaction may be carried out for a period of at least 20 minutes, preferably at least 40 minutes, and more preferably at least 1 hour. The polymerisation reaction may be carried out for a period of up 12 hours, preferably up to 8 hours, and more preferably up to 4 hours. Thus, the polymerisation reaction may be carried out for a period of from 20 minutes to 12 hours, preferably from 40 minutes to 8 hours, and more preferably from 1 hour to 4 hours.

The polymerisation reaction will typically be carried out in the presence of a catalyst, and preferably a basic catalyst.

The catalyst comprises may comprise titanium, tin, manganese, zinc, lead, nobelium, germanium, cobalt and/or antimony. In preferred embodiments, the catalyst is selected from antimony trioxide or antimony triacetate.

During polymerisation of BHET, a molecule of ethylene glycol is lost from each monomer. Thus, the method of the present invention preferably comprises removing ethylene glycol during the polymerisation reaction. This will typically be achieved by distillation. Where the method of the present invention comprises preparing the recycled BHET product, the ethylene glycol is preferably recycled to the series of depolymerisation reactors in step (a) of the PET recycling method.

The recycled BHET product will generally be passed to the polymerisation reactor in the form of a slurry or melt.

In some embodiments, the method comprises passing the recycled BHET product to a pre-polymerisation reactor before it is sent to the polymerisation reactor. Pre-polymerisation reactors are typically operated under milder conditions than polymerisation reactors, e.g. at lower temperature or under weaker vacuum, and preferably at lower temperature and under weaker vacuum. It will be appreciated that some polymerisation may occur during the pre-polymerisation reaction.

The pre-polymerisation reaction may be carried out at a temperature of at least 150° C., preferably at least 200° C., and more preferably at least 230° C. The pre-polymerisation reaction may be carried out at a temperature of up to 320° C., preferably up to 300° C., and more preferably up to 185° C. Thus, the pre-polymerisation reaction may be carried out at a temperature of from 150 to 320° C., preferably from 200 to 300° C., and more preferably from 230 to 285° C.

The pre-polymerisation reaction may be carried out at a pressure of from 0.1 to 101 kPa. Preferably the pre-polymerisation reaction may be carried out under vacuum, e.g. at a pressure of from 0.1 to 50 kPa.

In some embodiments, the method of the present invention may comprise further processing the polymer by extrusion, spinning, moulding and/or drawing. Drawing is particularly suitable for forming PET films, preferably by a process in which the polymer is drawn by being passed through a series of rollers.

For instance, the method may comprise moulding the polymer, e.g. into a bottle, packaging or textiles, and preferably into a clear bottle, such as a colour-free bottle.

The present invention also provides the use of a recycled BHET product in the preparation of a polymer comprising ethylene terephthalate monomer, wherein the recycled BHET product comprises isophthalic acid (IPA) in an amount of up to 0.5% by weight. The recycled BHET product, the polymer and/or the polymer preparation process are preferably as described herein. In some embodiments, the recycled BHET product may be used for simplifying a polymer preparation process.

The following non-limiting Examples illustrate the present invention.

### EXAMPLES

### Example 1: Depolymerisation Step (a)

Depolymerisation reactions in different series of reactors were simulated. The ratio of PET:ethylene glycol:catalyst system used in the simulation, by mass, was 1:4:0.005. Each reactor was simulated as operating at a temperature of 197° C., and at atmospheric pressure. The simulations were set so as to provide a conversion of 99.0% at the outlet of the final reactor in the series.

The results of the simulation are shown in the following table:

| | | Total | |
|---|---|---|---|
| Number of | Residence time | Residence | Conversion in |
| depolymerisation | per reactor | time | the outlet |
| reactors | (hours) | (hours) | (% starting PET) |
| 1 | 54 | 54 | (R1) 99.0% |
| 2 | 5 | 10 | (R1) 90.2% |
| | 5 | | (R2) 99.0% |
| 3 | 2 | 6 | (R1) 78.6% |
| | 2 | | (R2) 95.4% |
| | 2 | | (R3) 99.0% |
| 4 | 1.18 | 4.7 | (R1) 68.5% |
| | 1.18 | | (R2) 90.0% |
| | 1.18 | | (R3) 96.9% |
| | 1.18 | | (R4) 99.0% |
| 5 | 0.83 | 4.2 | (R1) 60.4% |
| | 0.83 | | (R2) 84.4% |
| | 0.83 | | (R3) 93.8% |
| | 0.83 | | (R4) 97.6% |
| | 0.83 | | (R5) 99.0% |

In order to obtain a production level of around 10,000 tonnes per year, the volume of a single reactor would be about 300 m³. Where a series of three reactors is used, the volume per reactor falls to just over 10 m³. A similar very large decrease in volume per reactor to approximately 11 to 12 m³ can be achieved with a series of only two reactors, as in the most preferred embodiments of the present invention.

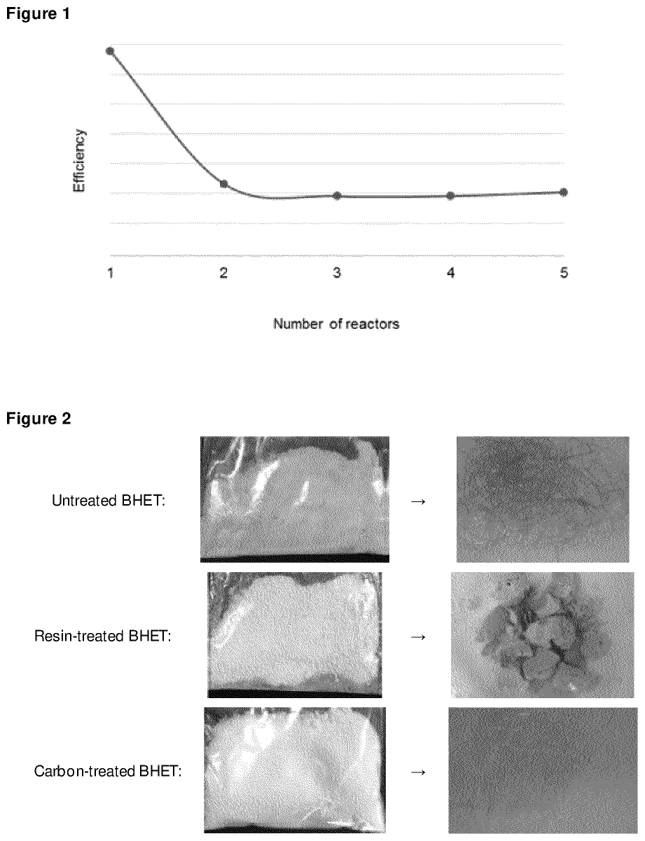

A graph showing the efficiency of each depolymerisation reaction, taking into account the data above but also energy and equipment input required in each arrangement, is shown in FIG. 1.

It can be seen that a dramatic improvement in efficiency is observed when a series of at least two depolymerisation reactors is used, as compared to the use of a single depolymerisation reactor.

### Example 2: Preferred Solvent for Use in Step (c)

BHET recrystallisation experiments were conducted in a variety of solvents, including methanol, ethanol, isopropanol, butanols and alcohols with a longer carbon chain.

Specifically, 50 g of crude BHET was dissolved in 250 ml of solvent at 80° C. for 1 hour. The BHET was recrystallised by cooling at a rate of 7° C./hour until a temperature of 10° C. was reached. The recrystallised BHET was analysed to determine its colour density. The weight loss during the recrystallisation processes was also measured.

The results are shown in the following table:

| | Weight loss | Colour density |
|---|---|---|
| Solvent | (%) | (b[h]) |
| Methanol | 28 | 4.06 |
| Ethanol | 57 | 4.05 |
| Isopropanol | 60 | 3.87 |
| Tert-butanol | — | 3.91 |
| N-butanol | 56 | 4.03 |
| N-heptanol | 22 | 4.80 |
| Octanol | 26 | 4.88 |
| Isooctanol | 27 | 6.07 |
| N-nonanol | 15 | 6.65 |

It can be seen that each of the lighter solvents gave good levels of decolouration. However, the amount of material lost during the recrystallisation was significantly lower in methanol than in any other of the lighter solvent experiments. Methanol, as well as higher alcohols, is viable for use on an industrial scale.

### Example 3: Decolourising Step (d)

A number of different techniques were used for decolourising an aqueous solution of BHET.

Experiments using resins gave promising results:

| Type of resin | Appearance of solution |
|---|---|
| Weak-acid cation exchange | High decolouration |
| Macroporous A | Moderate decolouration |
| Macroporous B | Good decolouration |
| Macroporous C | Good-moderate decolouration |
| Strong-acid cation exchange | Very high decolouration |
| Strong-base anion exchange | Moderate decolouration |
| Weak-base anion exchange A | Good-moderate decolouration |
| Weak-base anion exchange B | Good decolouration |

It can be seen that cation exchange resins, and particularly strongly acidic cation exchange resins, gave the most promising results.

Activated carbon was also highly effective at decolourising BHET:

| BHET sample | Colour density (b[h]) |
|---|---|
| Untreated | 7.21 |
| Cation exchange resin | 4.58 |
| Activated charcoal | 1.08 |

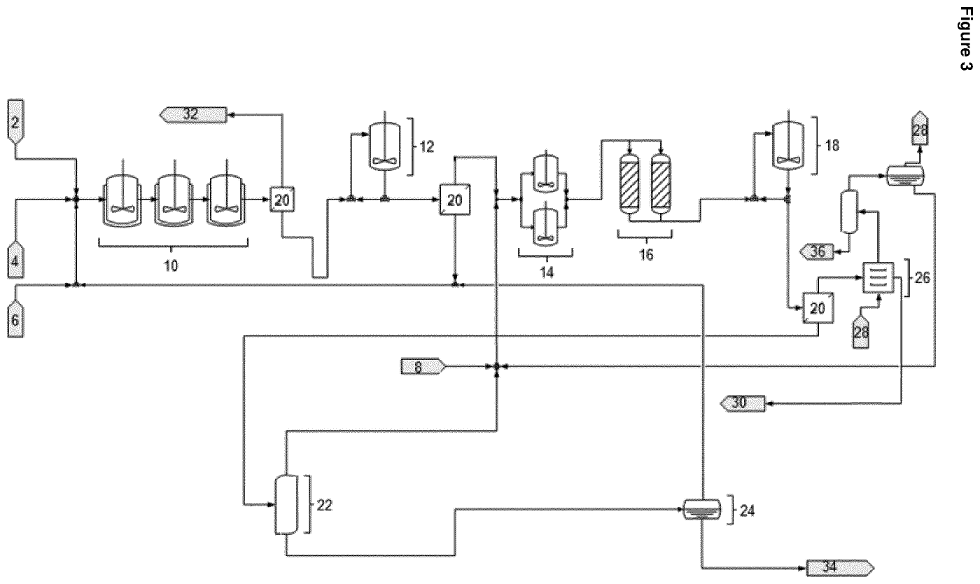

Pictures of the untreated and treated samples, and pictures of PET prepared using the samples, are shown in FIG. 2. While the cation exchange resin and active carbon both gave good levels of decolouration, the carbon-treated product gave a better quality polymer product.

Further decolourising experiments were carried out. This time, a solution of BHET in methanol was used. The experiments yielded similar results to those carried out on aqueous BHET solutions, but with cation exchange resins giving particularly good results.

### Example 4: Recycling Process Using Methanol in Step (c)

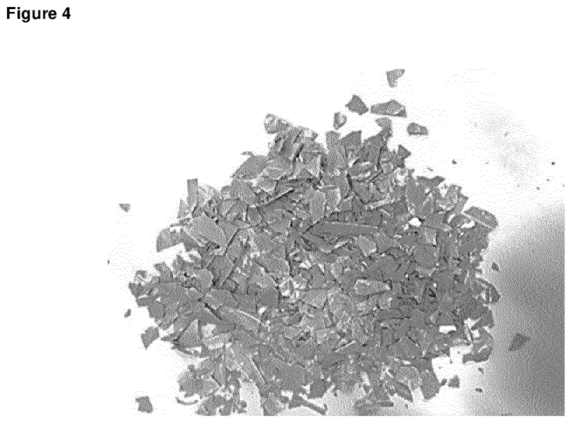

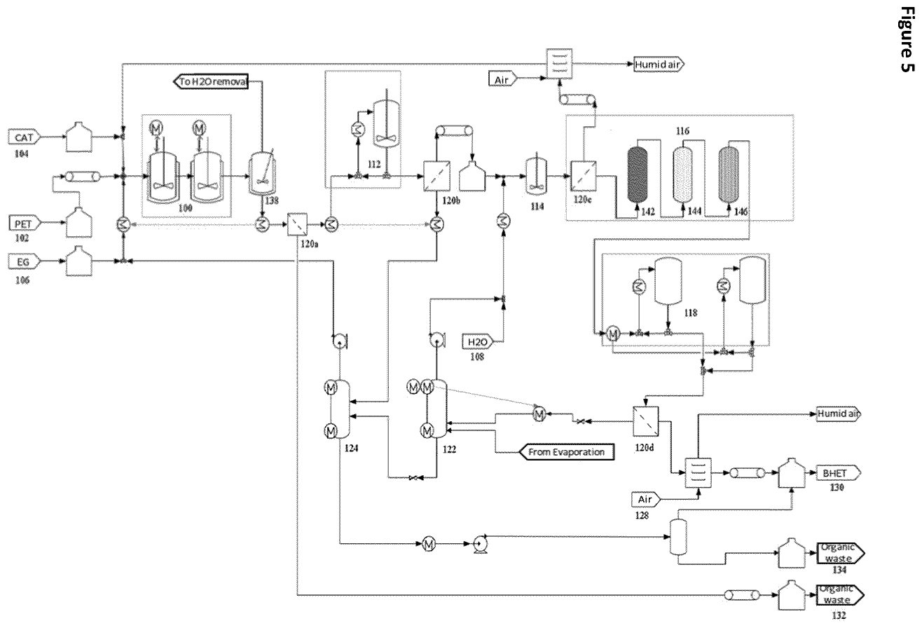

A process was carried out in the apparatus depicted in FIG. 3. Representative waste that was used in the process is shown in FIG. 4. The waste consists of blue and green used PET flakes.

Specifically, PET (2), a zinc acetate and urea catalyst system (4) and ethylene glycol (6) were passed to the first of a series of three depolymerisation reactors (10). A sample taken after the series of three depolymerisation reactors (10) showed 100% conversion of the PET (2) with 99.8% selectivity for BHET.

The depolymerised mixture was passed through a filter (20) to remove insoluble materials (32), then on to a crystalliser (12) in which a precipitate comprising BHET was formed. In this example, cooling crystallisation was used whereas evaporation crystallisation is preferred for the present invention. The precipitate was passed through a filter (20) to one of two stirred vessels (14).

Methanol (8) was added to the vessels (14) to dissolve the precipitate thereby forming a solution comprising BHET.

The solution was passed through a decolourisation stage (16), depicted in the picture as two units in parallel, to another crystalliser (18) where a purified product comprising BHET was formed.

The purified product was passed through another filter (20) to a drying unit (26), and the residual liquor passed to a methanol and ethylene glycol recovery unit (22). The methanol was recycled from recovery unit (22) to stirred vessels (14), while the ethylene glycol was passed through a flash unit (24), where organic waste (34) was removed, before being recycled to the series of depolymerisation reactors (10).

The purified product was dried by passing warm air (28) through drier (26). The warm air (28) was removed from the system via a condenser in which any waste water (36) is removed, and a flash unit from which methanol was recovered and recycled to stirred vessel (14). Once dried, the purified product (30) was removed from the system.

The purified product (30) had a low colour density and was used, without further processing, in the preparation of recycled PET for use in water bottles.

### Example 5: Recycling Process Using Water in Step (c)

A process was carried out in the apparatus depicted in FIG. 5.

Specifically, PET (102), a zinc acetate and urea catalyst system (104) and ethylene glycol (106) were passed to the first of a series of two depolymerisation reactors (100). A sample taken after the series of two depolymerisation reactors (100) showed 100% conversion of the PET (102), with selectivity for BHET at 95.0%; the other 5.0% of product consisted substantially of BHET oligomers.

Excess water (140) was removed by an evaporator (138), and the depolymerised mixture was then passed through a filter (120a) to remove insoluble materials (132), then on to a crystalliser (112) in which a precipitate comprising BHET was formed. In this example, cooling crystallisation was used whereas evaporation crystallisation is preferred for the present invention. The precipitate was passed through a filter (120b) to a stirred vessel (114).

Water (108) was added to the vessel (114) to dissolve the precipitate thereby forming a solution comprising BHET.

The solution was passed through a decolourisation stage (116). As depicted, the decolourisation stage comprises a filter (120c), followed by a first unit (142) comprising an activated carbon bed, followed in series by a second unit (144) comprising a cation exchange bed, and followed by a third unit (146) comprising an anion exchange bed. Following the decolourisation stage (116), the solution was passed to another crystalliser (118), in two stages, where a purified product comprising BHET was formed.

The purified product was passed through another filter (120d) to a drying unit (126), and the residual liquor passed to an evaporator (122). The water was recycled from the evaporator (122) to the stirred vessel (114), while the ethylene glycol was passed onwards to a further evaporator (124), where organic waste (134) was removed, before being recycled to the series of depolymerisation reactors (100).

The purified product was dried by passing warm air (128) through drier (126). Once dried, the purified product (130) was removed from the system.

The purified product (130) had a low colour density and was used, without further processing, in the preparation of recycled PET for use in water bottles.

### Example 6: Recycling Process Using Evaporation Crystallisation in Step (b) and Water in Step (c)

A depolymerisation process was simulated in an apparatus similar to that depicted in FIG. 5. A key difference was the use of a wiped film evaporator in place of cooling crystalliser (112).

Specifically, waste PET, a zinc acetate and urea catalyst system and ethylene glycol were passed to the first of a series of two depolymerisation reactors. The reactors were fitted with a reflux condenser to ensure that any vaporised ethylene glycol remained in the reactors. The reactors were operated at a temperature of 200° C. without the application of pressure. The duration of the depolymerisation reaction was 2.5 hours in total. Mass balance for the inlet and outlet of the series of depolymerisation of two depolymerisation reactors is as follows:

| | |
|---|---|
| Inlet | Outlet |

| | | | | |
|---|---|---|---|---|
| PET | 1478 | kg/hr | 15 | kg/hr |
| Ethylene glycol | 5911 | kg/hr | 5458 | kg/hr |
| Catalyst | 7.5 | kg/hr | 7.5 | kg/hr |
| BHET | 72 | kg/hr | 1984 | kg/hr |

| | | | |
|---|---|---|---|
| BHET oligomers | trace | 264 | kg/hr |

Other components were accounted for in the mass balance, but these were present in relatively minor amounts.

The mass balance shows almost complete depolymerisation of PET, with selectivity for BHET at approximately 98% in the depolymerised mixture.

Excess water was removed from the depolymerised mixture in a flash evaporator at a temperature of 200° C. and a pressure of 0.8 bar until a water content of 0.1% by weight was achieved. The depolymerised mixture was then passed through a centrifugal separator to remove waste solids, before being passed to an ethylene glycol evaporator.

The evaporator was operated at a temperature of 200° C. and a pressure of 0.1 bar. A precipitate comprising BHET was formed in the evaporator due to removal of a volatiles stream comprising ethylene glycol. Mass balance of the stream exiting the evaporator is as follows:

| | Outlet | |
|---|---|---|
| Ethylene glycol | 50 | kg/hr |
| BHET | 1953 | kg/hr |
| PET | trace | |
| BHET oligomers | 262 | kg/hr |

The stream containing the BHET precipitate was passed to a dissolution vessel, where water was added in an amount of 941 kg/hr to dissolve the precipitate thereby forming a solution comprising BHET. The dissolution vessel was operated at a temperature of 92° C. and without the application of pressure. The residence time in the dissolution vessel was 0.5 hours.

The solution comprising BHET was then passed through a centrifugal separator to remove any insoluble components such as BHET oligomers, before being passed to purification stage. In the purification stage, the solution comprising BHET was passed through a series of two activated carbon beds, followed by a series of two organic scavenger resins, followed by a series of two cation exchange resins, to form a purified solution comprising BHET.

Following the purification stage, the purified solution was passed to a crystalliser where a purified product comprising BHET was formed and subsequently dried. The purified BHET product (i.e. a recycled BHET product as described herein) contained 98.7% by weight BHET. Water from the crystalliser was recovered and recycled to the dissolution vessel.

### Example 7: Preparing PET from a Recycled BHET Product

A recycled BHET product was prepared using a method as described herein. The recycled BHET product was polymerised under standard conditions to form a recycled PET polymer having an IPA content of less than 0.2% by weight.

### Example 8: Preparation of PET from a Blended BHET Stream

PET is to be prepared from a monomer mixture having a target % by weight of IPA of 1.5%. This IPA level is desirable for preparing carbonated drinks bottles. A “dirty” BHET product, produced by conventional PET recycling processes, contains 2% by weight of IPA. Thus:

% IPA_{SecondBHET}=2%

% IPA_{Target}=1.5%

In order to provide a suitable monomer mixture for the PET, the “dirty” BHET product is blended with the recycled BHET product of Example 7 in a weight ratio of 0.75:0.25 to give a blended BHET stream. The blended BHET stream is polymerised under standard conditions to give a PET product with the desired properties.

1. A method for preparing a polymer comprising constitutional units derived from bis(2-hydroxyethyl) terephthalate (BHET), said method comprising carrying out a polymerisation reaction using a recycled BHET product comprising isophthalic acid (IPA), wherein the IPA is present in an amount of up to 0.5% by weight.

2. The method of claim 1, wherein the recycled BHET product comprises IPA in an amount of up to 0.2%, and more preferably up to 0.1%, by weight.

3. The method of any preceding claim, wherein the recycled BHET product is obtainable from a PET recycling process.

4. The method of claim 3, wherein the PET recycling process comprises:
(a) depolymerising PET in the presence of ethylene glycol and a catalyst system in a series of depolymerisation reactors to form a depolymerised mixture comprising bis(2-hydroxyethyl) terephthalate (BHET);
(b) crystallising a precipitate comprising BHET by removing a volatiles stream comprising ethylene glycol from the depolymerised mixture using evaporation crystallisation;
(c) dissolving the precipitate in a protic solvent to form a solution comprising BHET;
(d) removing impurities from the solution to form a purified solution comprising BHET; and
(e) crystallising a purified product comprising BHET from the purified solution,
wherein the purified product comprising BHET is the recycled BHET product.

5. The method of claim 4, wherein the method comprises providing the recycled BHET product by carrying out the PET recycling process.

6. The method of any preceding claim, wherein the amount of IPA present in the purified BHET is not measured prior to carrying out the polymerisation reaction.

7. The method of claim 5 or claim 6, wherein the amount of IPA that is present during the polymerisation reaction is not measured.

8. The method of any preceding claim, wherein the polymer is a polyethylene terephthalate (PET) homopolymer.

9. The method of any preceding claim, wherein the polymer is a PET copolymer.

10. The method of claim 9, wherein the PET copolymer is prepared from a monomer mixture containing the recycled BHET product in an amount of at least 25%, preferably at least 50%, and more preferably at least 90% by weight of monomers.

11. The method of claim 9 or claim 10, wherein the PET copolymer comprises constitutional units derived from IPA, diethylene glycol (DEG), butanediol (e.g. 1,4-butanediol), propanediol (e.g. 1,3-propanediol) and/or cyclohexanedimethanol (CHDM), and preferably wherein the PET copolymer comprises constitutional units derived from IPA.

12. The method of claim 11, wherein the polymer is prepared from a monomer mixture containing IPA in an amount of from 0.5 to 30%, preferably from 0.8 to 20%, and more preferably from 1 to 10% by weight of monomers.

13. The method of claim 11 or claim 12, wherein the IPA is added to the monomer mixture in an isolated form.

14. The method of any preceding claim, wherein the method comprises:
blending the recycled BHET product with a second BHET product to form a blended BHET stream; and
carrying out a polymerisation reaction on the blended BHET stream.

15. The method of claim 14, wherein the second BHET product is a recycled BHET product, preferably comprising IPA in an amount of at least 0.5%, preferably at least 0.8%, and more preferably at least 1% by weight.

16. The method of claim 14 or claim 15, wherein the blended BHET stream has a target % by weight IPA, % IPA_{Target}, and the first and second BHET products are blended in the weight ratio F:S, wherein:
F=1−S
S=% IPA_{Target}/% IPA_{SecondBHET}
where % IPA_{SecondBHET} represents the % by weight IPA in the second BHET product.

17. The method of any preceding claim, wherein the polymerisation reaction is carried out:
at temperature of from 200 to 350° C., preferably from 230 to 320° C., and more preferably from 250 to 300° C.;
under vacuum, for instance at a pressure of up to 80 kPa, preferably up to 10 kPa, and more preferably up to 1.0 kPa; and/or
for a period of from 20 minutes to 12 hours, preferably from 40 minutes to 8 hours, and more preferably from 1 hour to 4 hours.

18. The method of any preceding claim, wherein the polymerisation reaction is performed in the presence of a catalyst, preferably a basic catalyst.

19. The method of claim 18, wherein the catalyst comprises titanium, tin, manganese, zinc, lead, nobelium, germanium, cobalt and/or antimony, and preferably wherein the catalyst is selected from antimony trioxide or antimony triacetate.

20. The method of any preceding claim, wherein ethylene glycol is removed during the polymerisation reaction, preferably by distillation.

21. The method of any preceding claim, wherein the recycled BHET product is passed to the polymerisation reactor in the form of a slurry or melt.

22. The method of any preceding claim, wherein the method comprises carrying out a pre-polymerisation reaction under the following conditions:
at a temperature of from 230 to 285° C.; and/or
and a pressure of from 0.1 to 101 kPa.

23. The method of any preceding claim, wherein the method comprises further processing the polymer by extrusion, spinning, moulding and/or drawing.

24. The method of any preceding claim, wherein the method comprises moulding the polymer, e.g. into a bottle, packaging or textiles, and preferably into a clear bottle, such as a colour-free bottle.

25. Use of a recycled bis(2-hydroxyethyl) terephthalate (BHET) product comprising isophthalic acid (IPA) in the preparation of a polymer comprising ethylene terephthalate monomer, wherein the recycled BHET product comprises IPA in an amount of up to 0.5% by weight.

26. The use of claim 25, wherein the BHET and/or the polymer are as defined in any of claims 1 to 24.
